(19) United States
(12) Patent Application Publication
(10) Pub. No.: US 2026/0102455 A1
(43) Pub. Date: Apr. 16, 2026

(54) ANTIMICROBIAL HYDROLYZED COLLAGEN COMPOSITIONS AND METHODS OF USING THE SAME

(71) Applicant: Rochal Technologies, LLC, San Antonio, TX (US)

(72) Inventors: Rebecca E. McMahon, San Antonio, TX (US); Larry E. Estlack, San Antonio, TX (US); Bradley J. Rodier, San Antonio, TX (US); Jonathan D. Velasco, San Antonio, TX (US); Tiffany F. Sanchez, Helotes, TX (US); Sergio A. Medina, San Antonio, TX (US); Fahimeh Tabatabaei, Boerne, TX (US); Ann Beal Salamone, Gainesville, FL (US); Nina Bionda, Ithaca, NY (US)

(73) Assignee: Rochal Technologies, LLC, San Antonio, TX (US)

(21) Appl. No.: 19/354,492

(22) Filed: Oct. 9, 2025

Related U.S. Application Data
(60) Provisional application No. 63/705,729, filed on Oct. 10, 2024.

(51) Int. Cl. A61K 38/01; A61K 31/047; A61K 31/155; A61K 47/18; A61P 31/04; A61P 31/10

(52) U.S. Cl. CPC A61K 38/014; A61K 31/047; A61K 31/155; A61K 47/183; A61P 31/04; A61P 31/10

(57) ABSTRACT

A composition is provided that includes hydrolyzed collagen, at least one biguanide, at least one vicinal diol, and at least one chelating agent. The composition exhibits at least 2-log reduction of planktonic microbes within 24 hours. Methods of using the composition are also provided.

### CROSS-REFERENCE TO RELATED APPLICATIONS

This application claims priority to U.S. Provisional Patent Application No. 63/705,729, filed Oct. 10, 2024, the entirety of which is incorporated by reference herein.

### FIELD OF INVENTION

This invention relates generally to an antimicrobial hydrolyzed collagen-based composition with at least one biguanide, at least one hydrophobic vicinal diol, and at least one chelating agent which is effective in the treatment for healing of acute and chronic wounds.

### BACKGROUND

Hydrolyzed collagen is derived from collagen (approximately 300,000 Da) that has been denatured to break collagen's triple helix structure and partially hydrolyzed to form gelatin. Further protein molecular weight reduction occurs through hydrolysis of gelatin (acid, base, and/or enzyme) to produce hydrolyzed collagen with a possible molecular weight range of from 50 to 12,000 Da. Amino acids typically found in hydrolyzed collagen are alanine, arginine, aspartate, glutamate, glycine, histidine, isoleucine, leucine, lysine, methionine, phenylalanine, proline, hydroxyproline, serine, threonine, tyrosine and valine—plus the presence of glutamic acid and aspartic acid. Glycine, proline and hydroxyproline are concentration predominant in hydrolyzed collagen. Hydrolyzed collagen is readily water soluble from cold to hot temperatures and does not gel (Bloom strength of zero) except at high % solids (e.g., 60 wt % or higher in aqueous media).

Hydrolyzed collagen is used in a broad variety of applications, such as a dietary supplement to support skin, nail, hair, joint, bone, and muscle health; a wound healing agent; a surgical incision site healing agent; and a cosmetic ingredient. It is commercially available in many forms, such as powder, liquid, gel, capsule, foam, and spray.

### SUMMARY OF THE INVENTION

In one aspect, a hydrolyzed collagen (HC) composition (also referred to as “composition” or “antimicrobial composition” or “hydrolyzed collagen antimicrobial composition”) is provided that includes a hydrolyzed collagen, at least one biguanide, at least one vicinal diol, and at least one chelating agent. The hydrolyzed collagen composition has antimicrobial properties to mitigate infection while retaining biocompatibility. The hydrolyzed collagen composition can exhibit at least a 2-log reduction of planktonic microbes.

In some embodiments of the hydrolyzed collagen composition, hydrolyzed collagen is present at a concentration of 10 wt-% to 99.9 wt-%; at least one biguanide is present in a concentration of 0.01 wt-% to 1 wt-%; at least one vicinal diol is present at a concentration of 0.04 wt-% to 4 wt-%; and at least one chelating agent is present at a concentration of 0.008 wt-% to 1 wt-%, wherein all percentages are based on total weight of the hydrolyzed collagen composition.

The hydrolyzed collagen compositions described herein can be used to reduce or eliminate microbes in food and nutritional supplements.

The hydrolyzed collagen compositions described herein can be used in cosmetics and personal care products.

The hydrolyzed collagen compositions described herein can be used to treat surgical incision sites.

The hydrolyzed collagen compositions described herein can be used to treat impaired soft tissue and hard tissue.

The hydrolyzed collagen compositions described herein can be used to treat acute and chronic wounds, as well as burn wounds.

The hydrolyzed collagen compositions described herein can be used to reduce and/or eliminate Gram-positive and Gram-negative bacteria in or on wounds, tissues, surfaces and devices.

The hydrolyzed collagen compositions described herein can be used to reduce and/or eliminate fungi in or on wounds, tissues, surfaces and devices.

The hydrolyzed collagen compositions described herein can be used to reduce and/or eliminate yeast in or on wounds, tissues, surfaces and devices.

The hydrolyzed collagen compositions described herein can be used to reduce and/or eliminate mold in or on wounds, tissues, surfaces and devices.

The hydrolyzed collagen compositions described herein can be used to reduce and/or eliminate viruses in or on wounds, tissues, surfaces and devices.

The hydrolyzed collagen compositions described herein can be used to facilitate tissue healing by incorporation of hydrolyzed collagen and at least one biguanide, at least one hydrophobic vicinal diol and at least one chelator.

The hydrolyzed collagen compositions described herein can provide compositions that are non-cytotoxic to mammalian cells.

The hydrolyzed collagen compositions described herein can be deposited on a surface or a device to reduce and/or eliminate microbes while providing biocompatibility.

These and other objectives and advantages of the antimicrobial compositions described herein, some of which are specifically described and others that are not, will become apparent from the detailed description and claims that follow.

### BRIEF DESCRIPTION OF THE DRAWINGS

FIG. 1 is a bar chart showing the results of a cytotoxicity assay plotted as % viability compared to untreated samples, where the PC line is the positive control and the NC line is the negative control.

FIG. 2 is a bar chart showing the diameter of the zone of clearance of microbial growth.

### DETAILED DESCRIPTION OF THE INVENTION

In some aspects, compositions including hydrolyzed collagen, at least one biguanide, at least one vicinal diol, and at least one chelating agent are described. In some embodiments, the composition exhibits at least a 2-log reduction of planktonic microbes. In some embodiments, the composition exhibits at least a 3-log, at least 4-log, at least 5-log, and at least 6-log reduction of planktonic microbes. In some embodiments, the reduction is measured at 30 minutes or 1 hour. In some embodiments, the reduction is measured at 24 hours. In some embodiments, the planktonic microbes are at least one of Gram-positive bacteria (e.g., methicillin-resistant Staphylococcus aureus (MRSA)), Gram-negative bacteria (e.g., Pseudomonas aeruginosa), or fungi (e.g., Candida albicans).

Hydrolyzed collagen may be derived from acid, alkaline, enzymatic, or combination methods for hydrolysis of collagen (molecular weight of about 300 kDa) to produce hydrolyzed collagen (molecular weight of generally less than 12 kDa). The source of collagen can be from human, bovine, porcine, piscine, ovine, or avian sources, but can include other sources and can be a mixture of collagen sources.

In some embodiments, the hydrolyzed collagen is at a concentration of 10 wt % to 99.9 wt %, or 60 wt % to 99.9 wt %, or 80 wt % to 99.9 wt %, based on total weight of the composition.

The biguanide can be a polymeric biguanide. In some embodiments, the at least one biguanide is present at a concentration of 0.01 wt % to 1 wt %, based on the total weight of the composition. In some embodiments, the at least one biguanide is present at a concentration of 0.075 wt % to 0.5 wt %, or 0.1 wt % to 0.15 wt % based on the total weight of the composition.

In some embodiments, the at least one biguanide can include a bis(biguanide). In some embodiments, the bis(biguanide) can be alexidine and its salts, chlorhexidine and its salts, or a combination thereof. In some embodiments, the bis(biguanide) can be present in a concentration from 0.001 wt % to 0.035 wt % based on the total weight of the composition.

In some embodiments, the at least one biguanide comprises poly(hexamethylene biguanide) (PHMB).

In some embodiments, the vicinal diol may be a monoalkyl glycol, glycerol alkyl ether, and monoacyl glycerol. In some embodiments, the vicinal diol is a hydrophobic vicinal diol.

In some embodiments, the at least one vicinal diol is present in a concentration from 0.04 wt % to 4 wt %, or 0.1 wt % to 1 wt %, or 0.4 wt % to 0.6 wt % based on total weight of the composition.

In some embodiments, the at least one vicinal diol comprises glycerol 1-(2-ethylhexyl) ether, 1,2-dihydroxyoctane, or a combination of both.

In some embodiments, the chelating agent is present at a concentration of from 0.008 wt % to 1 wt %. In some embodiments, the chelating agent is present at a concentration of from 0.025 wt % to 0.5 wt %, or from 0.05 wt % to 0.2 wt % based on the total weight of the composition.

In some embodiments, the composition includes:
10 wt-% to 99.9 wt-% of hydrolyzed collagen;
0.01 wt-% to 1 wt-% of at least one biguanide;
0.04 wt-% to 4 wt-% of at least one vicinal diol; and
0.008 wt-% to 1 wt-% of at least one chelating agent, wherein all percentages are based on total weight of the composition.

In some embodiments, the composition comprises at least one polymeric biguanide or polymeric bis(biguanide) or at least one low molecular weight bis(biguanide). This combination of antimicrobial biguanides may enhance efficacy against the number and type of pathogenic microbial species.

A preferred polymeric biguanide is poly(hexamethylene biguanide) (PHMB), commercially available under the trademark Cosmocil™ CQ. The hexamethylene biguanide polymers are also referred to as poly(hexamethylene biguanide) (PHMB), poly(hexamethylene bisbiguanide) (PHMB), poly(hexamethylene guanide) (PHMB), poly(aminopropyl biguanide) (PAPB), poly[aminopropyl bis(biguanide)](PAPB), polyhexanide and poly(iminoimidocarbonyl)iminohexamethylene hydrochloride. PHMB is the preferred abbreviation for this biocidal polymer. PHMB is a broad-spectrum antimicrobial and has been used in contact lens multipurpose solutions, wound rinsing solutions, wound dressings, perioperative cleansing products, mouthwashes, surface disinfectant, food disinfectant, veterinary applications, cosmetic preservative, paper preservative, secondary oil recovery disinfectant, industrial water treatment, and in swimming pool cleaners. PHMB is normally obtained in the hydrochloride form in water.

Preferred low molecular weight bis(biguanides) for antimicrobial activity include alexidine (ALEX) and chlorhexidine (CHG). Alexidine often exists in the dihydrochloride form while chlorhexidine is often in its gluconate form. Alexidine is also listed chemically as 1,1′-hexamethylenebis[5-(2-ethylhexyl)biguanide]dihydrochloride. Chlorhexidine gluconate is also listed chemically as 1,1′-hexamethylenebis[5-(p-chlorophenyl)biguanide] di-D-gluconate. Chlorhexidine has been used in many biomedical applications, while alexidine has been used primarily in mouthwash and in contact lens solutions.

Biguanide salts include, but are not limited to, gluconates, nitrates, acetates, phosphates, sulfates, halides and the like.

In some embodiments, the hydrolyzed collagen composition can include at least one antimicrobial hydrophobic vicinal diol selected from a hydrophobic monoalkyl glycol, a hydrophobic glycerol alkyl ether, and a hydrophobic monoacyl glycerol. In addition to being branched or unbranched, these compounds can either be saturated or unsaturated.

In some embodiments, preferred hydrophobic vicinal diols, in particular monoalkyl glycols, can be selected from caprylyl glycol (also known as 1,2-dihydroxyoctane, 1,2-octanediol, and 1,2-octylene glycol), hexylene glycol, 2-methyl-2,4-pentanediol, 1,3-butylene glycol, triethylene glycol, and glycol bis(hydroxyethyl) ether, with caprylyl glycol (1,2-dihydroxyoctane), being more preferred, and caprylyl glycol, a component of Sensiva® SC 10 with glycerol 1-(2-ethylhexyl) ether (2-ethylhexylglycerin), being most preferred.

Sensiva® SC 10 is reported to combine the excellent skin care and deodorizing properties of 2-ethylhexylglycerin (Sensiva® SC 50) with the moisturizing and antimicrobial properties of caprylyl glycol. Additionally, it can contribute to the antimicrobial stability of cosmetic formulations. It can also be used to improve the efficacy of traditional cosmetic preservatives, such as parabens or phenoxyethanol (Schülke & Mayr, Sensiva® SC 10 Multifunctional Cosmetic Ingredient). Screening tests with Sensiva® SC 10 have shown that it reliably inhibits the growth and multiplication of Gram-positive odor causing bacteria, while at the same time it does not affect beneficial skin flora. The antimicrobial efficacy of methylparaben preservative is accelerated by Sensiva® SC 10 in reduction of CFU/mL for Aspergillus niger (ATCC 6275), Candida albicans (ATCC 10231), Staphylococcus aureus (ATCC 6538), Pseudomonas aeruginosa (ATCC 15442) and Escherichia coli (ATCC 11229). The concentrations used were 0.2 (wt) % methylparaben, 1.0 (wt) % Sensiva® SC 10, and a combination of 0.2 (wt) % methylparaben and 1.0 (wt) % Sensiva® SC 10. The recommended preservative use concentration for Sensiva® SC 10 by Schülke & Mayr is 0.5-2.0%.

In some embodiments, the glycerol alkyl ethers (hydrophobic) can be selected from 1-O-heptylglycerol, 1-O-octylglycerol, 1-O-nonylglycerol, 1-O-decylglycerol, 1-O-undecylglycerol, 1-O-dodecylglycerol, 1-O-tridecylglycerol, 1-O-tetradecylglycerol, 1-O-pentadecylglycerol, 1-O-hexadecylglycerol (chimyl alcohol), 1-O-heptadecylglycerol, 1-O-octadecylglycerol (batyl alcohol), 1-O-octadec-9-enyl glycerol (selachyl alcohol), glycerol 1-(2-ethylhexyl) ether (also known as octoxyglycerin, 2-ethylhexyl glycerin, 3-(2-ethylhexyloxy)propane-1,2-diol, and Sensiva® SC 50), 2-ethylhexyl diglycol ether, 2-ethylhexyl oligoglycol ethers, glycerol 1-heptyl ether, glycerol 1-octyl ether, glycerol 1-decyl ether, and glycerol 1-dodecyl ether, glycerol 1-tridecyl ether, glycerol 1-tetradecyl ether, glycerol 1-pentadecyl ether, glycerol 1-hexadecyl ether and glycerol 1-octadecyl ether. More preferred glycerol alkyl ethers include glycerol 1-(2-ethylhexyl) ether (Sensiva® SC 50) and 1-O-dodecylglycerol. In some embodiments, the glycerol alkyl ether is a glycerol 1-(2-ethylhexyl) ether.

Sensiva® SC 50 can reliably inhibit Gram-positive odor-causing bacteria on the skin and is used in deodorant formulations. It is reported to boost the efficacy of traditional preservatives.

In some embodiments, the monoacyl glycerols (hydrophobic) can be selected from 1-O-decanoylglycerol (monocaprin), 1-O-undecanoylglycerol, 1-O-undecenoylglycerol, 1-O-dodecanoylglycerol (monolaurin, also called glycerol monolaurate and Lauricidin®), 1-O-tridecanoylglycerol, 1-O-tetradecanoylglycerol (monomyristin), 1-O-pentadecanoylglycerol, 1-O-hexadecanoylglycerol, 1-O-heptadecanoylglycerol, and 1-O-octanoylglycerol (monocaprylin), with 1-O-decanoylglycerol, 1-O-dodecanoylglycerol, and 1-O-tetradecanoylglycerol being preferred. In some embodiments, the monoacyl glycerols (hydrophobic) can include 1-O-octanoylglycerol. In some embodiments, the monoacyl glycerols (hydrophobic) can include 1-O-dodecanoylglycerol. In some embodiments, glycerols substituted in the 1-O-position are more preferred than those substituted in the 2-O-position, or disubstituted in the 1-0 and 2-0 positions.

Since the antimicrobial hydrophobic monoalkyl vicinal diol, and an antimicrobial hydrophobic monoalkyl and monoacyl glycerol have hydrophilic —OH groups but low or negligible water solubility, it is preferred that a surfactant be added to aid in solution compatibilization and homogeneity of these compounds.

Chelating agents enhance the susceptibility of bacteria and other organisms to the biocidal effects of the antimicrobial agent, thus rendering an antimicrobial composition containing a chelating agent more effective in combating infection. Additionally, chelating agents deactivate matrix metalloproteases (MMPs), enzymes that can impede tissue formation and healing by breaking down collagen. MMPs are often found at elevated levels in compromised, inflamed tissue. Chelating agents bind to zinc ions, which are necessary for MMP activity, disrupting the MMP, causing deactivation, and thus facilitating collagen development and healing.

In some embodiments, the chelating agent is selected from any compound that is able to sequester monovalent or polyvalent metal ions, such as sodium, lithium, rubidium, cesium, calcium, magnesium, barium, cerium, cobalt, copper, iron, manganese, nickel, strontium or zinc, and is pharmaceutically or veterinary acceptable. The outermost surface of bacterial cells universally carries a net negative charge, which is usually stabilized by divalent cations such as Mg²⁺ and Ca²⁺. This is associated with the teichoic acid and polysaccharide elements of Gram-positive bacteria, the lipopolysaccharide of Gram-negative bacteria, and the cytoplasmic membrane itself. Thus, the chelating agent aids in destabilizing microorganisms.

In some embodiments, suitable chelating agents comprise, but are not limited to, amino carboxylic acids, citric acid, ethylenediaminetetraacetic acid (EDTA), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N′-bis(2-hydroxybenzyl)ethylenediamine-N,N′-diacetic acid, ethylenediamine-N,N-diacetic acid, ethylenediamine-N,N-dipropionic acid, triethylenetetraaminehexaacetic acid, ethylenediamine-N,N′-bis(methylenephosphonic acid), iminodiacetic acid, NN-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid and biphosphonates such as editronate, and salts thereof. Suitable chelating agents include, for example but are not limited to, hydroxyalkylphosphonates as disclosed in U.S. Pat. No. 5,858,937, specifically the tetrasodium salt of 1-hydroxyethylidene-1,1-diphosphonic acid, also referred to as tetrasodium etidronate, commercially available as DeQuest 2016 diphosphonic acid sodium salt or phosphonate.

In some embodiments, preferred chelating agents are mixed salts of EDTA such as disodium, trisodium, tetrasodium, dipotassium, tripotassium, tetrapotasssium, lithium, dilithium, ammonium, diammonium, triammonium, tetraammonium, calcium and calcium-disodium. In some embodiments, the EDTA salts are disodium, trisodium or tetrasodium salts of EDTA. In some embodiments, the EDTA salts are disodium EDTA and/or trisodium EDTA.

The composition can also include a surfactant. Suitable surfactants include, but are not limited to, cationic, anionic, nonionic, amphoteric and ampholytic surfactants. Preferred surfactants are nonionic and amphoteric surfactants. The surfactants can have an HLB (hydrophilic-lipophilic balance) value of 18-30 in order to maintain the biocidal activity of the antimicrobial agents, while facilitating a non-cytotoxic composition. The surfactant lowers surface tension, facilitating wetting of a surface for enhanced activity of the biocidal agent and for assistance with debridement.

Suitable nonionic surfactants include the ethylene oxide/propylene oxide block copolymers of poloxamers, reverse poloxamers, poloxamines, and reverse poloxamines. Poloxamers and poloxamines are preferred, and poloxamers are most preferred. Poloxamers and poloxamines are available from BASF Corp. under the trade names of Pluronic® and Tetronic®.

In some embodiments, suitable Pluronic surfactants comprise but are not limited to Pluronic F38 having a HLB of 31 and average molecular weight (AMW) of 4,700, Pluronic F68 having a HLB of 29 and AMW of 8,400, Pluronic 68LF having a HLB of 26 and AMW or 7,700, Pluronic F77 having a HLB of 25 and AMW of 6,600, Pluronic F87 having a HLB of 24 and AMW of 7,700, Pluronic F88 having a HLB of 28 and AMW or 11,400, Pluronic F98 having a HLB of 28 and AMW of 13,000, Pluronic F108 having a HLB of 27 and AMW of 14,600, Pluronic F127 (also known as Poloxamer 407) having a HLB of 18-23 and AMW of 12,600, and Pluronic L35 having a HLB of 19 and AMW of 1,900.

Another class of surfactants is that of the diamino block copolymers of ethylene oxide and propylene oxide sold under the trade name Tetronic®. An exemplary surfactant of this type is Tetronic 1107 (also known as Poloxamine 1107).

In some embodiments, surfactants that may be added include polyethylene glycol esters of fatty acids, e.g., coconut, polysorbate, polyoxyethylene or polyoxypropylene ethers of higher alkanes (C12-C18), polysorbate 20 available under the trademark Tween 20, polyoxyethylene (23) lauryl ether available under the trademark Brij 35, polyoxyethylene (40) stearate available under the trademark Myrj 52, and polyoxyethylene (25) propylene glycol stearate available under the trademark Atlas G 2612. Other neutral surfactants include nonylphenol ethoxylates such as nonylphenol ethoxylates, Triton X-100, Brij surfactants of polyoxyethylene vegetable-based fatty ethers, Tween 80, decyl glucoside, and lauryl glucoside.

Examples of amphoteric surfactants suitable for use in antimicrobial compositions according to the present invention include materials of the type offered commercially under the trademark Miranol. Another useful class of amphoteric surfactants is exemplified by cocoamidopropyl betaine, commercially available from various sources.

Where the hydrolyzed collagen antimicrobial composition is an aqueous solution or may be hydrated endogenously or exogenously, a water-soluble polymer can be added to increase solution viscosity, to change rheology, and to prolong residence time of the antimicrobial composition on a biological surface or medical device.

In some embodiments, it is often desirable to include water-soluble viscosity builders in the hydrolyzed collagen antimicrobial compositions of the present invention. Because of their demulcent effect and possible hydrophobic interactions with biological tissue, water-soluble polymers have a tendency to enhance the interaction with a biological tissue by means of a hydrated film on the surface. Because of this behavior, such water-soluble polymers can increase the residence time of the hydrolyzed collagen antimicrobial composition on a biological tissue. Aqueous media may be incorporated into the composition or may be derived from a treatment surface that is wet with, for example, wound exudate.

Examples of water-soluble viscosity builders include, but are not limited to, methylcellulose, hydroxyethylcellulose, hydroxypropylcellulose, hydroxypropylmethylcellulose, carboxymethylcellulose, polyquaternium-1, polyquaternium-6, polyquaternium-10, guar, hydroxypropylguar, hydroxypropylmethylguar, cationic guar, carboxymethylguar, hydroxymethylchitosan, hydroxypropylchitosan, carboxymethylchitosan, N-[(2-hydroxy-3-trimethylammonium)propyl]chitosan chloride, water-soluble chitosan, hyaluronic acid and its salts, chondroitin sulfate, heparin, dermatan sulfate, amylose, amylopectin, pectin, locust bean gum, alginate, dextran, carrageenan, xanthan gum, gellan gum, scleroglucan, schizophyllan, gum arabic, gum ghatti, gum karaya, gum tragacanth, pectins, starch and its modifications, tamarind gum, poly(vinyl alcohol), poly(ethylene oxide), poly(ethylene glycol), poly(methyl vinyl ether), polyacrylamide, poly(N,N′-dimethylacrylamide), poly(N-vinylacetamide), poly(N-vinylformamide), poly(2-hydroxyethyl methacrylate), poly(glyceryl methacrylate), poly(N-vinylpyrrolidone), poly(dimethylaminoethyl methacrylate), poly(dimethylaminopropyl acrylamide), polyvinylamine, poly(N-isopropylacrylamide) and poly(N-vinylcaprolactam), the latter two hydrated below their Lower Critical Solution Temperatures, and the like, and combinations thereof.

If anionic hydrophilic polymers are utilized for enhancing viscosity, the overall polymer negative charge may electrostatically attract and accumulate the cationic biguanides and a greater concentration of biguanide will then be needed to provide biocidal efficacy comparable to the utilization of a neutral or cationic water-soluble polymer. Thus, preferred water soluble polymers are neutral in charge, such as hydroxyethylcellulose, hydroxypropylcellulose, hydroxypropylmethylcellulose, guar, hydroxypropylguar, hydroxypropylmethylguar, poly(ethylene oxide), and poly(N-vinylpyrrolidone), or cationic in charge, such as cationic chitosans, cationic cellulosics, and cationic guar. Chitosan polymers may also enhance the antimicrobial behavior of the hydrolyzed collagen antimicrobial composition. More preferred hydrophilic polymers comprise hydroxypropylmethylcellulose, hydroxypropylcellulose, hydroxypropylguar, hydroxymethylchitosan, poly(ethylene oxide), N-[(2-hydroxy-3-trimethylammonium)propyl]chitosan chloride, with hydroxymethylpropylcellulose being most preferred.

In some embodiments, when the hydrolyzed collagen antimicrobial composition includes an aqueous media, the pH can be adjusted to between 4.5 to 7.0, with pH 5.0 to pH 6.5 being more preferred, and pH 5.5 to 6.0 being most preferred. The role of wound bed pH is of fundamental importance during the healing of chronic wounds, and prolonged acidification of the wound bed has been shown to increase the healing rate in chronic venous leg ulcers (Wilson et al, 1979) and many other examples.

Suitable buffers to adjust and maintain pH can include sodium citrate, potassium citrate, citric acid, sodium dihydrogen phosphate, disodium monophosphate, boric acid, sodium borate, tartrate, phthalate, succinate, acetate, propionate, maleate salts, tris(hydroxymethyl)aminomethane, amino alcohol buffers, and buffers such as ACES, PIPES, and MOPOSO, and mixtures thereof. One or more buffers can be added to solutions of the present invention in amounts ranging between approximately 0.01 to 2.0 weight percent by volume, but more preferably between approximately 0.05 to 0.5 weight percent by volume.

Additionally, the pH of the hydrolyzed collagen antimicrobial composition can be adjusted by the combination of ethylenediaminetetracetic acid (EDTA) disodium and trisodium salt chelating agents, with this method being most preferred.

In some embodiments, emollients/moisturizers and humectants can be added to the antimicrobial formulation to provide a more soothing hydrolyzed collagen antimicrobial composition when used topically. Emollients/moisturizers function by forming an oily layer on the top of the skin that traps water in the skin. Petrolatum, lanolin, mineral oil, dimethicone, and siloxy compounds are common emollients. Other emollients include isopropyl palmitate, isopropyl myristate, isopropyl isostearate, isostearyl isostearate, diisopropyl sebacate, propylene dipelargonate, 2-ethylhexyl isononoate, 2-ethylhexyl stearate, cetyl lactate, lauryl lactate, isopropyl lanolate, 2-ethylhexyl salicylate, cetyl myristate, oleyl myristate, oleyl stearate, oleyl oleate, hexyl laurate, and isohexyl laurate, lanolin, olive oil, cocoa butter, shea butter, octyldodecanol, hexyldecanol, dicaprylyl ether and decyl oleate.

Examples of humectants include, but are not limited to, glycerin, lecithin, 1,2-propylene glycol, dipropylene glycol, polyethylene glycol, 1,3-butylene glycol, and 1,2,6-hexanetriol. Humectants function by drawing water into the outer layer of skin.

In some embodiments, the hydrolyzed collagen composition can also include wetting agents, buffers, gelling agents or emulsifiers. Other excipients could include various water-based buffers ranging in pH from 5.0-7.5, silicones, polyether copolymers, vegetable and plant fats and oils, vitamins, laurate esters, myristate esters, palmitate esters, and stearate esters.

In some embodiments, additional anti-inflammatory agents can be added to the hydrolyzed collagen composition, such as water-soluble derivatives of aspirin, vitamin C, methylsulfonylmethane, tea tree oil, and non-steroidal anti-inflammatory drugs.

In some embodiments, essential oils can also be added to the hydrolyzed collagen composition as fragrance or aromatic agents, and/or as antimicrobial agents, including thymol, menthol, sandalwood, camphor, cardamom, cinnamon, jasmine, lavender, geranium, juniper, menthol, pine, lemon, rose, eucalyptus, clove, orange, mint, linalool, spearmint, peppermint, lemongrass, bergamot, citronella, cypress, nutmeg, spruce, tea tree, wintergreen (methyl salicylate), vanilla, and the like. More preferred essential oils include thymol, sandalwood oil, wintergreen oil and eucalyptol for antimicrobial properties and pine oil for fragrance.

In some embodiments, one or more biologically active agents may be incorporated into the hydrolyzed collagen antimicrobial composition to provide a medical benefit to a living host. Examples of biologically active agents that can be incorporated into the hydrolyzed collagen composition include, but are not limited to, cells, stem cells, amniotic tissue, amniotic cells, exosomes, growth factors, decellularized extracellular matrix derived from stem cells, micronized decellularized tissue, granulated crosslinked bovine tendon collagen and glycosaminoglycans, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, manuka honey, topical retinoids, hormones, tumor-specific antibodies, antisense oligonucleotides, small interfering RNA (siRNA), anti-VEGF RNA aptamer, nucleic acids, DNA, DNA fragments, DNA plasmids, Si-RNA, transfection agents, vitamins, essential oils, liposomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-tluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle.

In some embodiments, the hydrolyzed collagen antimicrobial composition includes at least one of cells, stem cells, amniotic tissue, amniotic cells, exosomes, growth factors, decellularized extracellular matrix derived from stem cells, micronized decellularized tissue, granulated collagen, gelatin, or glycosaminoglycans. In some embodiments, the cells can be animal cells. In some embodiments, the cells can be mammalian cells. In some embodiments, the cells can be non-mammalian cells.

In some embodiments, the hydrolyzed collagen antimicrobial composition includes at least one additional ingredient selected from glycolipids, glycoproteins, immunological response modifiers, saccharides, and polysaccharides.

In some embodiments, the hydrolyzed collagen antimicrobial composition may be delivered in different forms. Exemplary forms include, but not limited to, powders, films, sheets, liquids, creams, pastes, foams, lotions, gels, emulsions, suspensions, tablets, capsules, sprays and aerosols. The hydrolyzed collagen antimicrobial composition can also be imbibed by swabs, cloth, sponges, foams, wound dressing materials and non-woven and paper products, such as paper towels and wipes. Formulations of the subject hydrolyzed collagen antimicrobial compositions may additionally comprise organic solvents, emulsifiers, gelling agents, moisturizers, stabilizers, time release agents, dyes, and like components commonly employed in formulations for body administration.

In some embodiments, the hydrolyzed collagen antimicrobial compositions may also be added to catheters, and other medical devices, in a hydrated or dried form to provide a coating that can be inserted into a body in order to prevent microbial attachment to the catheter, and other medical devices.

Alternatively, in some embodiments, the hydrolyzed collagen antimicrobial composition may be added to a solid or porous support, such as a polymeric foam, a polymer film, a woven, knitted or nonwoven material, and then dried and applied directly to a tissue or medical device. In some embodiments, a treated porous support, such as a polymeric foam, may also absorb tissue exudate, creating a hydrated environment for controlled release of hydrolyzed collagen, polylysine, or other additives.

### Methods

In another aspect, a method of treating tissue comprising contacting the tissue with a hydrolyzed collagen antimicrobial composition as described herein is provided.

In some embodiments, the tissue being treated is impaired tissue. In some embodiments, the impaired tissue comprises at least one of inflamed tissue, diseased tissue (e.g., cancer), a cut, a wound, a lesion, a rash, a fistula, a burn, a void, a surgical site, a diabetic (foot) ulcer, a venous ulcer, a pressure ulcer, a tissue affected by cellulitis, dehisced wounds, necrotic wounds, traumatic wounds with foreign bodies (i.e., puncture wounds), or a medical implant site.

In some embodiments, the method includes contacting the tissue on a periodic schedule. For example, at least once a day, at least twice a day, or at least three times a day. In some embodiments, the periodic treatment can continue for at least two days, at least three days, at least four days, at least five days, at least six days, or at least seven days.

In some embodiments, the hydrolyzed collagen composition is applied subcutaneously. In some embodiments, the hydrolyzed collagen composition is applied via injection. In some embodiments, the injection can be intradermal, subcutaneous, oral, intramuscular, submucosal, intranasal, vaginal, buccal, intrathecal, epidural, intraparenchymal, ocular, subretinal, dental, intra-tumoral, intracardiac, intra-articular, intravenous, intracavernous, intraosseous, intraperitoneal, intra-abdominal, intra-fascial, intra-organ, and intravitreal.

In some embodiments, the hydrolyzed collagen composition is applied topically. In some embodiments, the hydrolyzed collagen composition is applied using a syringe (with or without applicator or delivery tip) or tube.

### EXPERIMENTAL

The following materials and abbreviations are used in the experimental section.
HC: Hydrolyzed collagen, Peptiplus XB, Gelita, lot 8621390-082322
PHMB: (Poly(hexamethylene biguanide))—dry, Rochal, 52-86-d
EDTA-Di: (Ethylenediaminetetraacetic acid disodium salt), JT Baker, lot H225593
EDTA-Tri: (Ethylenediaminetetraacetic acid trisodium salt), Spectrum Chemicals, lot 1DK0688
SC 50: (Sensiva® SC 50, Glycerol 1-(2-ethylhexyl) ether), Schülke & Mayr, lot 1178933
SC 10: (Sensiva® SC 10, 1,2-Dihydroxyoctane), Schülke & Mayr, lot 1203109
NaCl: sodium chloride, EMID, lot K39163704A

### Example 1: Antimicrobial Efficacy

Hydrolyzed collagen powder was blended with powders of PHMB, SC 50, SC 10, EDTA-Di, EDTA-Tri and NaCl to form a uniform blended powder (Table 1) and tested for planktonic antimicrobial efficacy.

TABLE 1
Hydrolyzed collagen with antimicrobials (HC + AM) composition

| Chemicals | wt % | grams |
|---|---|---|
| HC | 98.69 | 24.676 |
| PHMB* | 0.1 | 0.025 |
| SC10* | 0.1 | 0.025 |
| SC50* | 0.3 | 0.076 |
| EDTA-Di* | 0.06 | 0.014 |
| EDTA-Tri* | 0.02 | 0.004 |
| NaCl* | 0.73 | 0.184 |

*Component of antimicrobial (AM) composition

Testing of the blended powder (HC+AM) shown in Table 1 was conducted by adding 1 gram of powder to a sterile cup to which 0.1 mL of bacteria or fungi were added directly to the powder. The cup was swirled to mix and then incubated at 25° C. for 24 hours. The microbial-containing powder was added to 9 mL of Dey-Engley (DE) buffer and vortexed. The aliquot of the resulting mixture (0.1 mL) was then plated onto agar and incubated for 24 hours at 37° C. Colonies were enumerated to determine log₁₀ CFU/mL.

Additional testing was conducted using the blended powder (0.1 g) of Table 1 dissolved in 10 mL of sterile phosphate-buffered saline (PBS) to produce a 10 wt % solution (9.8 wt % HC, 0.01 wt % PHMB, 0.04 wt % vicinal diol, 0.008 wt % EDTA salts). To this solution, 0.1 mL of bacteria or fungi were added, swirled to mix, and incubated at 25° C. for 24 hours. Then, 1 mL of the microbe-containing solution was added to 9 mL of DE buffer and vortexed to mix. The resulting solution (0.1 mL) was then plated onto agar and incubated for 24 hours at 37° C. Colonies were enumerated to determine log₁₀ CFU/mL.

The dry powder formulation and the 1 wt % saline solution formulation were tested for antimicrobial activity using three microorganisms (methicillin-resistant S. aureus (MRSA), P. aeruginosa, and C. albicans) at 30 minutes and 60 minutes (solution formulations only) and 24-hour treatment times. Hydrolyzed collagen, saline, and antimicrobials (without HC) were the controls. Colonies were enumerated and log reduction determined.

With MRSA, complete kill was found at 24 hours with either the HC+AM solution or powder and corresponded to using the antimicrobials alone in solution (AM solution) which also produced complete kill at 24 hours (Table 2). At 30 and 60 minutes, the HC+AM solution and the AM solution demonstrated complete kill of MRSA, while the HC+AM powder showed about 3.1 log reduction of the initial load in 30 min, and complete kill in 60 min. Hydrolyzed collagen by itself demonstrated negligible antimicrobial effectiveness against MRSA in this experimental set up.

TABLE 2
MRSA log reduction

| Test article | 30 minutes | 60 minutes | 24 hours |
|---|---|---|---|
| HC solution | −0.061 | 0.029 | 0.002 |
| HC + AM solution | 6.117 | 6.037 | 6.136 |
| HC powder | 0.069 | 0.079 | 0.123 |
| HC + AM powder | 3.115 | 6.212 | 6.136 |
| AM solution | 6.117 | 6.037 | 6.136 |
| PBS | 0 | 0 | 0 |

With P. aeruginosa, complete kill was found at 24 hours with either the HC+AM solution or powder and corresponded to using the AM solution which also produced complete kill at 24 hours (Table 3). At 30 minutes, the AM solution demonstrated complete kill of P. aeruginosa while the HC+AM solution and HC+AM powder demonstrated 3-log and 2-log reduction, respectively. However, by 60 minutes treatment time, the HC+AM solution demonstrated complete kill while the HC+AM powder resulted in about 3-log reduction. Hydrolyzed collagen by itself demonstrated negligible antimicrobial effectiveness against P. aeruginosa in this experimental set up.

TABLE 3
P. aeruginosa log reduction

| Test article | 30 minutes | 60 minutes | 24 hours |
|---|---|---|---|
| HC solution | 0.041 | 0.053 | −0.162 |
| HC + AM solution | 3.18 | 6.030 | 6.008 |
| HC powder | 0.033 | −0.047 | −0.144 |
| HC + AM powder | 2.112 | 2.950 | 6.008 |
| AM solution | 6.1 | 6.030 | 6.008 |
| PBS | 0 | 0 | 0 |

With C. albicans, the HC+AM powder, the HC+AM solution, and the AM solution produced complete kill at 24 hours. With 30 minutes of treatment, the AM solution produced complete kill while the HC+AM solution and HC+AM powder yielded 2-log reduction of C. albicans. At 60 minutes treatment time both had slightly increased antimicrobial efficacy. Hydrolyzed collagen by itself demonstrated negligible antimicrobial effectiveness against C. albicans in this experimental set up.

TABLE 4
C. albicans log reduction

| Test article | 30 minutes | 60 minutes | 24 hours |
|---|---|---|---|
| HC solution | 0.095 | 0.179 | 0.012 |
| HC + AM solution | 1.944 | 2.383 | 6.101 |
| HC powder | 0.067 | 0.063 | −0.157 |
| HC + AM powder | 2.105 | 3.260 | 6.101 |
| AM solution | 6.103 | 6.121 | 6.101 |
| PBS | 0 | 0 | 0 |

### Example 2: Anti-Biofilm Efficacy

Biofilms were prepared by diluting stocks of microorganisms (methicillin-resistant S. aureus, P. aeruginosa, and C. albicans) to 6×10⁵ CFU/mL. 50 μL of this stock was added to each well of a polypropylene 96-well plate followed by addition of 150 μL of tryptic soy broth (TSB). The plates were incubated at 37° C. for 24 hours. After the designated incubation time, the wells were aspirated, gently washed once with phosphate buffered saline (PBS) (200 μL/well) followed by addition of treatment (200 μL/well). The HC+AM treatment was prepared by dissolving 1 g of HC+AM powder in 9 mL of water. The plate was then incubated at 25° C. for 24 hours (h), then the treatments were removed and the wells once again washed gently with PBS. To each of the wells 200 μL of Dey Engley neutralizing broth was added and the plate was sonicated for 30 min followed by serial dilution and plating of samples for evaluation of reduction in biofilm-associated microorganisms.

Reduction in biofilm-associated microbial load is summarized in Table 5.

TABLE 5
Log reduction in biofilm-associated microbial
load presented as log₁₀(CFU/mL)

| | HC + AM | PBS control |
|---|---|---|
| | (CFU/mL) | (CFU/mL) |
| Microorganism | Log Reduction | Log Reduction |
| MRSA | 1.64 | 0 |
| P. aeruginosa | 1.68 | 0 |
| C. albicans | 1.58 | 0 |

### Example 3: Biocompatibility Testing

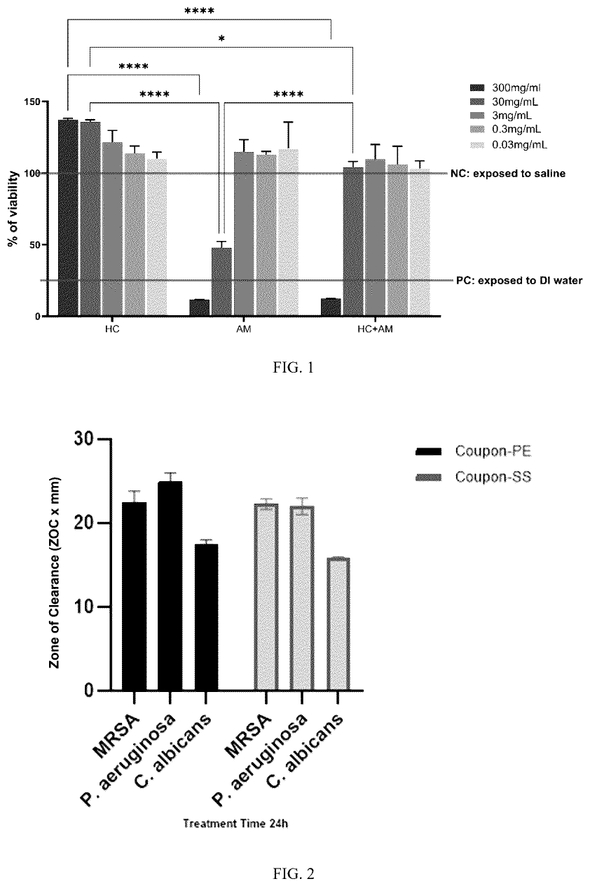

The cytotoxicity of the hydrolyzed collagen+antimicrobial formulation (HC+AM) was compared to hydrolyzed (HC) alone and antimicrobial (AM) composition alone using primary normal human dermal fibroblasts (HDFa, Cat. No. PCS:201-012 Lot: 80902232 from ATCC, Manassas, VA) and assessing cell recovery after 24 hours of exposure. Cells were seeded in Dulbecco's Modified Eagle Medium (DMEM) supplemented with 2% fetal bovine serum (Cat. No, 11885-084 Lot: 3047861) at 7,500 cells per well in a 96-well plate and incubated overnight at 37° C. under a humidified atmosphere with 5% CO₂. To treat the cells, a stock solution of HC was prepared at 300 mg/mL in serum-free media (SFM), followed by 1:10 dilutions in the same media. To enable direct comparison, both AM and HC+AM treatments, which contain 0.1% PHMB, were normalized to the same final PHMB concentration of 300 μg/mL. For AM alone, this was achieved by mixing 3 mL of AM with 7 mL of SFM. For HC+AM, 3 g of HC+AM powder was dissolved in 10 mL of SFM, which also yielded a final PHMB concentration of 300 μg/mL. This normalization ensured that any observed differences in cell viability can be attributed to the presence of HC rather than differences in PHMB content. Following overnight (˜16 h) incubation, the medium was gently aspirated, cells washed with buffered saline, and 100 μL of test solution added to each well. The cells were incubated for 2 min with the treatments which were then aspirated, and the cells were washed with buffered saline. Fresh medium was added to the wells and then the cells were incubated for 24 hours. After a 24 hours recovery time, alamarBlue assay was performed to determine viability of the treated cells. A 1:10 dilution of alamarBlue reagent (cat. No: K2243, Batch No: 8) was prepared using phenol red-free cell culture medium (DMEM). To each well of the 96-well plate, 100 μL of the alamarBlue working solution was added and the plates were incubated for 1 hour at 37° C. After the incubation, the plate was read for fluorescence measurement (excitation of 560 nm and emission of 590 nm). The data is shown in FIG. 1 as % viability determined based on the viability readouts of the untreated samples (negative control).

While none of the concentrations of the antimicrobial formulation led to complete eradication of the cells, the presence of hydrolyzed collagen improved cell viability. The difference was especially noticeable at 30 mg/mL concentration. This could be related to the observed slower antimicrobial performance of the HC+AM formulations and shows potential benefits of combining an antimicrobial formulation with a hydrolyzed collagen in balancing the antimicrobial activity and cytotoxicity towards mammalian cells.

### Example 4: Composition Deposition

RD128-316HP Stainless Steel (316L; SS) and polyethylene (PE) coupons (½″ or 12.7 mm in diameter) were coated with 0.5 g of a gel formulation of HC+AM (65 wt % total of solids in water) for 2 hours at 25° C. The coupons were then placed onto a freshly inoculated agar plate of MRSA, P. aeruginosa or C. albicans and the plates were incubated at 37° C. for 24 h. After incubation, the zone of inhibition (ZOI) was measured using ImageJ. The results are shown in FIG. 2. The values provided are in mm and refer to a diameter of the zone of clearance of microbial growth. The overall zones ranged from 16.6 to 27 mm in diameter with the smallest zones being for C. albicans and the largest zones for P. aeruginosa.

The HC+AM formulation (6.5 g of powder+3.5 g of sterile water) was heated to 37° C. until it became flowable, then applied to a sterile 4″×4″, 12-ply cotton gauze pad and allowed to cool to room temperature. Post-application, the gauze exhibited slight tackiness, attributable to the HC+AM gel, allowing it to adhere to itself and the surface without permanently bonding. The gauze remained removable from surfaces and could be unfolded without tearing.

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. A composition comprising hydrolyzed collagen, at least one biguanide, at least one vicinal diol, and at least one chelating agent.

2. The composition of claim 1, wherein the composition exhibits at least 2-log reduction of planktonic microbes within 24 hours.

3. The composition of claim 1, wherein the hydrolyzed collagen is present at a concentration of 10 wt-% to 99.9 wt-% based on a total weight of the composition.

4. The composition of claim 1, wherein the at least one biguanide comprises a polymeric biguanide.

5. The composition of claim 1, wherein the at least one biguanide is present in a concentration of 0.01 wt-% to 1 wt-% based on the total weight of the composition.

6. The composition of claim 1, wherein the at least one biguanide comprises a bis(biguanide).

7. The composition of claim 6, wherein the bis(biguanide) is present at a concentration of 10 ppm to 350 ppm based on the total weight of the composition.

8. The composition of claim 1, wherein the at least one biguanide comprises poly(hexamethylene biguanide (PHBMB).

9. The composition of claim 1, wherein the vicinal diol is at least one of a monoalkyl glycol, a glycerol alkyl ether, or a monoacyl glycerol.

10. The composition of claim 9, wherein the at least one vicinal diol is present at a concentration of 0.04 wt-% to 4 wt-% based on total weight of the composition.

11. The composition of claim 9, wherein the at least one vicinal diol comprises glycerol 1-(2-ethylhexyl) ether, 1,2-dihydroxyoctane, or a combination of both.

12. The composition of claim 1, wherein the at least one chelating agent is present at a concentration of 0.008 wt-% to 1 wt-% based on total weight of the composition.

13. The composition of claim 12, wherein the at least one chelating agent is selected from the group consisting of aminocarboxylic acids, citric acid, ethylenediaminetetraacetic acid (EDTA), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N-bis(2-hydroxybenzyl)ethylenediamine-N,N-diacetic acid, ethylenediamine-N,N-diacetic acid, ethylenediamine-N,N-dipropionic acid, triethylenetetraaminehexaacetic acid, ethylenediamine-N,N′-bis(methylenephosphonic acid), iminodiacetic acid, N,N-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid, bisphosphonates, or salts thereof.

14. The composition of claim 12, wherein the at least one chelating agent is ethylenediaminetetracetic acid (EDTA) or its salts.

15. The composition of claim 1, wherein:
hydrolyzed collagen is present at a concentration of 10 wt-% to 99.9 wt-%;
at least one biguanide is present in a concentration of 0.01 wt-% to 1 wt-%;
at least one vicinal diol is present at a concentration of 0.04 wt-% to 4 wt-%; and
at least one chelating agent is present at a concentration of 0.008 wt-% to 1 wt-%, wherein all percentages are based on total weight of the composition.

16. The composition of claim 1, further comprising a biologically active agent selected from cells, stem cells, amniotic tissue, amniotic cells, exosomes, growth factors, decellularized extracellular matrix derived from stem cells, micronized decellularized tissue, granulated crosslinked bovine tendon collagen and glycosaminoglycans, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, manuka honey, topical retinoids, hormones, tumor-specific antibodies, antisense oligonucleotides, small interfering RNA (siRNA), anti-VEGF RNA aptamer, nucleic acids, DNA, DNA fragments, DNA plasmids, Si-RNA, transfection agents, vitamins, essential oils, liposomes, exosomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-tluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle.

17. The composition of claim 1, further comprising at least one of cells, stem cells, amniotic tissue, amniotic cells, exosomes, growth factors, decellularized extracellular matrix derived from stem cells, micronized decellularized tissue, granulated collagen, gelatin, or glycosaminoglycans.

18. The composition of claim 1, further comprising at least one additional ingredient selected from glycolipids, glycoproteins, immunological response modifiers, saccharides, and polysaccharides.

19. The composition of claim 1, wherein the composition has a form selected from a powder, a liquid, a gel, a paste, a cream, a suspension, an emulsion, a film, a sheet, a foam, a lotion, a spray, an aerosol, a capsule, or a tablet.

20. A method of treating tissue comprising contacting the tissue with a composition of claim 1.
